(19) United States
(12) Patent Application Publication
(10) Pub. No.: US 2021/0125723 A1
(43) Pub. Date: Apr. 29, 2021

(54) METHOD FOR PROCESSING RESULT DATA OF MEDICAL EXAMINATION

(71) Applicants: SAMSUNG SDS CO., LTD., Seoul (KR); SAMSUNG LIFE PUBLIC WELFARE FOUNDATION, Seoul (KR)

(72) Inventors: Yong Seok LEE, Seoul (KR); Min Young LEE, Seoul (KR); Yong Min PARK, Seoul (KR); Young Hyuck IM, Gyeonggi-do (KR); Jong Han YU, Seoul (KR); Se Kyung LEE, Seoul (KR); Ju Hee CHO, Seoul (KR); Dan Bee KANG, Seoul (KR); Mi Ra KANG, Seoul (KR); Seok Jin NAM, Seoul (KR); Seok Won KIM, Seoul (KR); Jeong Eon LEE, Seoul (KR); Jai Min RYU, Seoul (KR); Ji Yeon KIM, Seoul (KR); Soo Yong SHIN, Seoul (KR)

(21) Appl. No.: 17/081,290

(22) Filed: Oct. 27, 2020

(30) Foreign Application Priority Data
Oct. 28, 2019 (KR) 10-2019-0134637

(51) Int. Cl. G16H 50/20; G06N 3/08; A61B 6/00; A61B 8/08; A61B 5/00; A61B 5/145

(52) U.S. Cl. CPC G16H 50/20; G06N 3/08; A61B 6/502; A61B 5/14546; A61B 5/7275; A61B 5/14532; A61B 8/0825

(57) ABSTRACT

A method for processing medical examination data performed by a computing device, including determining a time interval that is last increased in a first repeating as an optimal time interval, configuring a feature matrix having a time axis according to the optimal time interval, and by using the feature matrix, setting a look-back window size to a predetermined initial size to obtain a performance evaluation result of the trained RNN-based model, second repeating increasing the look-back window size and then obtaining a second performance evaluation result of the RNN-based model trained according to the increased look-back window size until the second performance evaluation result is no longer improved, determining the look-back window size that is last increased in the second repeating as an optimal look-back window size and training the RNN-based model according to the optimal look-back window size by using the feature matrix having the optimal time interval.

### CROSS REFERENCE TO RELATED APPLICATIONS AND CLAIM OF PRIORITY

This application claims the benefit of Korean Patent Application No. 10-2019-0134637, filed on Oct. 28, 2019, in the Korean Intellectual Property Office, the disclosure of which is incorporated herein by reference in its entirety.

### BACKGROUND

### 1. Field

The present inventive concept relates to a method for training an artificial neural network that receives result data of a medical examination and predicts a future progress of an examinee, and a method for predicting a future progress of an examinee depending on a result of a medical examination of the examinee using a trained artificial neural network.

### 2. Description of the Related Art

Artificial intelligence technology is used in various fields such as medical fields. For example, there have been attempts to predict a future progress of an examinee by analyzing a result of medical examination of the examinee. Here, it is advantageous for accurate prediction to analyze results of multiple sequential medical examinations rather than results of just one medical examination. However, a time interval for the result data of the medical examination may vary depending on circumstances of the examinee.

### SUMMARY

Aspects of the inventive concept provide a method for machine learning based on a recurrent neural network (RNN), in which the method is robust in situations where the examinee's examination time is not constant, and targets data of a medical examination in the form of time series data, and a method for predicting a future progress of an examinee using a model trained through the method for machine learning.

Aspects of the inventive concept also provide a method for machine learning of breast cancer recurrence prediction model using examination data after breast cancer surgery, and a method for predicting a possibility of recurrence of breast cancer in an examinee using a model trained through the method or machine learning.

However, aspects of the inventive concept are not restricted to the one set forth herein. The above and other aspects of the inventive concept will become more apparent to one of ordinary skill in the art to which the inventive concept pertains by referencing the detailed description of the inventive concept given below.

According to an aspect of the inventive concept, there is provided a method for processing medical examination data, wherein the medical examination data is time series data, and wherein the method is performed by a computing device, and comprises setting a time interval applied to a time axis of a two-dimensional feature matrix comprising the time axis and each feature as a predetermined initial interval to configure the feature matrix, and obtaining a first performance evaluation result of a recurrent neural network (RNN)-based model trained by using the feature matrix, first repeating increasing the time interval and then obtaining the first performance evaluation result of the RNN-based model trained by using the feature matrix according to the increased time interval until the first performance evaluation result is no longer improved, determining the time interval that is last increased in the first repeating as an optimal time interval, configuring the feature matrix with the time axis according to the optimal time interval, and by using the feature matrix, setting a look-back window size to a predetermined initial size to obtain a performance evaluation result of the trained RNN-based model, second repeating increasing the look-back window size and then obtaining a second performance evaluation result of the RNN-based model trained according to the increased look-back window size until the second performance evaluation result is no longer improved, determining the look-back window size that is last increased in the second repeating as an optimal look-back window size and training the RNN-based model according to the optimal look-back window size by using the feature matrix having the optimal time interval.

According to another aspect of the inventive concept, there is provided a method, wherein obtaining the first performance evaluation result comprises training the RNN-based model by setting the look-back window size to a predetermined default size, and, wherein the first repeating comprises training the RNN-based model by setting the look-back window size to the predetermined default size

According to another aspect of the inventive concept, there is provided a method, wherein obtaining the first performance evaluation result comprises filling a missing value according to the initial interval by using a regression model generated with data of a time slot in which the medical examination data exists, and wherein the first repeating comprises filling the missing value according to the initial interval by using the regression model generated with data of the time slot in which the medical examination data exists.

According to another aspect of the inventive concept, there is provided a method, wherein the RNN-based model outputs data related to prediction of breast cancer recurrence after breast cancer surgery, and, wherein features included within the feature matrix comprises mammography category, ultrasonography category, albumin level, absolute lymphocyte count (ALC) level, absolute neutrophil count (ANC) level, alkaline phosphatase (ALP) level, alanine aminotransferase (ALT) level, aspartate aminotransferase (AST) level, total bilirubin level, calcium level, total cholesterol level, glucose level, hemoglobin level, total protein level, white blood cell (WBC) level, carcinoembryonic antigen (CEA) level, and CA 15-3 level.

According to another aspect of the inventive concept, there is provided a method, wherein the features included within the feature matrix further comprises radiotherapy category, chemotherapy category, hormonal therapy category, and target therapy category after breast cancer surgery.

According to another aspect of the inventive concept, there is provided a method, wherein the features included within the feature matrix further comprises synchronous contralateral cancer category, whether there is lymphatic invasion or not, whether there is NAC (Nipple Areola Complex) involvement or not, tumor stage, lymph nodes, whether it is estrogen receptor positive or not, whether it is progesterone receptor positive or not, whether it is HER2 (human epidermal growth factor receptor 2) positive or not, whether it is CK56 positive or not, whether it is EGFR (Epidermal Growth Factor Receptor) positive or not, Ki67(%) category, and preoperative CA 15-3 (Cancer Antigen 15-3) level.

According to another aspect of the inventive concept, there is provided a method, wherein the RNN-based model outputs data related to prediction of breast cancer recurrence after breast cancer surgery, and, wherein the method further comprises, after training the RNN-based model according to the optimal look-back window size by using the feature matrix having the optimal time interval, inputting latest examination data of an examinee into the trained RNN-based model and obtaining data for predicting breast cancer recurrence, and, wherein the latest examination data comprise the number of latest examination data corresponding to the optimal look-back window size of the examinee.

According to another aspect of the inventive concept, there is provided a method, wherein the medical examination data is time series data, and wherein the method is performed by a computing device, and comprises obtaining latest examination data of an examinee, and configuring a feature matrix by using the latest examination data, and inputting the feature matrix into an RNN-based model, and generating data for predicting breast cancer recurrence of the examinee by using an output value of the RNN-based model, wherein feature included within the feature matrix comprises mammography category, ultrasonography category, albumin level, absolute lymphocyte count (ALC) level, absolute neutrophil count (ANC) level, alkaline phosphatase (ALP) level, alanine aminotransferase (ALT) level, aspartate aminotransferase (AST) level, total bilirubin level, calcium level, total cholesterol level, glucose level, hemoglobin level, total protein level, white blood cell (WBC) level, carcinoembryonic antigen (CEA) level, CA 15-3 level, radiotherapy category, chemotherapy category, hormonal therapy category, target therapy category after breast cancer surgery, synchronous contralateral cancer category, whether there is lymphatic invasion or not, whether there is NAC involvement or not, tumor stage, lymph nodes, whether it is estrogen receptor positive or not, whether it is progesterone receptor positive or not, whether it is HER2 positive or not, whether it is CK56 positive or not, whether it is EGFR positive or not, Ki67(%) category, and preoperative CA 15-3 level.

According to another aspect of the inventive concept, there is provided a method, wherein a time axis of the feature matrix includes time slots having a predetermined optimal time interval are sequentially connected by a number corresponding to a predetermined optimal look-back window size, and wherein configuring the feature matrix comprises filling a missing value due to not performing a medical examination corresponding to the time slot of the feature matrix by using a regression model generated with data of the time slot in which the medical examination data exists.

According to another aspect of the inventive concept, there is provided an apparatus for processing examination data, wherein the apparatus comprises a processor, a memory, and a computer program loaded into the memory and executed by the processor, the computer program comprising, an instruction for setting a time interval applied to a time axis of a two-dimensional feature matrix comprising the time axis and each feature as a predetermined initial interval to configure the feature matrix, and obtaining a first performance evaluation result of a recurrent neural network (RNN)-based model trained by using the feature matrix, an instruction for first repeating increasing the time interval and then obtaining the first performance evaluation result of the RNN-based model trained by using the feature matrix according to the increased time interval until the first performance evaluation result is no longer improved, an instruction for determining the time interval that is last increased in the instruction for first repeating as an optimal time interval, an instruction for configuring the feature matrix with the time axis according to the optimal time interval, and by using the feature matrix, setting a look-back window size to a predetermined initial size to obtain a performance evaluation result of the trained RNN-based model, an instruction for second repeating increasing the look-back window size and then obtaining a second performance evaluation result of the RNN-based model trained according to the increased look-back window size until the second performance evaluation result is no longer improved, an instruction for determining the look-back window size that is last increased in the instruction for second repeating as an optimal look-back window size, and an instruction for training the RNN-based model according to the optimal look-back window size by using the feature matrix having the optimal time interval.

According to another aspect of the inventive concept, there is provided an apparatus, wherein the instruction for obtaining the first performance evaluation result comprises an instruction for training the RNN-based model by setting the look-back window size to a predetermined default size, and wherein the instruction for first repeating comprises an instruction for training the RNN-based model by setting the look-back window size to the predetermined default size.

According to another aspect of the inventive concept, there is provided an apparatus, wherein the instruction for obtaining the first performance evaluation result comprises an instruction for filling a missing value according to the initial interval by using a regression model generated with data of a time slot in which the medical examination data exists, and wherein the instruction for first repeating comprises the instruction for filling the missing value according to the initial interval by using the regression model generated with data of the time slot in which the medical examination data exists.

According to another aspect of the inventive concept, there is provided an apparatus, wherein the RNN-based model outputs data related to prediction of breast cancer recurrence after breast cancer surgery, and wherein features included within the feature matrix comprises mammography category, ultrasonography category, albumin level, absolute lymphocyte count (ALC) level, absolute neutrophil count (ANC) level, alkaline phosphatase (ALP) level, alanine aminotransferase (ALT) level, aspartate aminotransferase (AST) level, total bilirubin level, calcium level, total cholesterol level, glucose level, hemoglobin level, total protein level, white blood cell (WBC) level, carcinoembryonic antigen (CEA) level, and CA 15-3 level.

According to another aspect of the inventive concept, there is provided an apparatus, wherein the features included within the feature matrix further comprises radiotherapy category, chemotherapy category, hormonal therapy category, and target therapy category after breast cancer surgery.

According to another aspect of the inventive concept, there is provided an apparatus, wherein the features included within the feature matrix further comprises synchronous contralateral cancer category, whether there is lymphatic invasion or not, whether there is NAC involvement or not, tumor stage, lymph nodes, whether it is estrogen receptor positive or not, whether it is progesterone receptor positive or not, whether it is HER2 positive or not, whether it is CK56 positive or not, whether it is EGFR positive or not, Ki67(%) category, and preoperative CA 15-3 level.

According to another aspect of the inventive concept, there is provided an apparatus, wherein the RNN-based model outputs data related to prediction of breast cancer recurrence after breast cancer surgery, and wherein the computer program further comprises, after the instruction for training the RNN-based model according to the optimal look-back window size by using the feature matrix having the optimal time interval, an instruction for inputting latest examination data of an examinee into the trained RNN-based model and obtaining data for predicting breast cancer recurrence, and wherein the latest examination data comprise the number of latest examination data corresponding to the optimal look-back window size of the examinee.

### BRIEF DESCRIPTION OF THE DRAWINGS

These and/or other aspects will become apparent and more readily appreciated from the following description of the embodiments, taken in conjunction with the accompanying drawings in which:

FIG. 1 is a block diagram of a system for analyzing examination data according to an embodiment of the present inventive concept;

FIG. 2 is a flow chart of a method for analyzing examination data according to another embodiment of the present inventive concept;

FIG. 3 is a flowchart for explaining in more detail some operations of the method for analyzing the examination data according to the embodiment described with reference to FIG. 2;

FIG. 4 is a diagram for explaining a configuration of a feature matrix of medical examination data referenced in some embodiments of the present inventive concept;

FIG. 5 is a diagram for explaining a process of restoring missing values of medical examination data in some embodiments of the present inventive concept;

FIG. 6 is a flowchart for explaining in more detail some other operations of the method for analyzing the examination data according to the embodiment described with reference to FIG. 2;

FIG. 7 is a flowchart for explaining a process in which a feature included in a feature matrix referred to in some embodiments of the present inventive concept is selected from among a plurality of feature candidates;

FIGS. 8A to 8B are diagrams illustrating a list of 33 features used to learn an RNN-based model for predicting breast cancer recurrence and to predict breast cancer recurrence using the trained RNN-based model, in some embodiments of the present inventive concept;

FIG. 9 is a diagram showing the accuracy of an RNN-based breast cancer recurrence prediction model generated according to some embodiments of the present inventive concept; and

FIG. 10 is a hardware configuration diagram of an exemplary computing device capable of implementing a device according to some embodiments of the present inventive concept.

### DETAILED DESCRIPTION

Hereinafter, preferred embodiments of the present disclosure will be described with reference to the attached drawings. Advantages and features of the present disclosure and methods of accomplishing the same may be understood more readily by reference to the following detailed description of preferred embodiments and the accompanying drawings. The present disclosure may, however, be embodied in many different forms and should not be construed as being limited to the embodiments set forth herein. Rather, these embodiments are provided so that this disclosure will be thorough and complete and will fully convey the concept of the disclosure to those skilled in the art, and the present disclosure will only be defined by the appended claims.

In adding reference numerals to the components of each drawing, it should be noted that the same reference numerals are assigned to the same components as much as possible even though they are shown in different drawings. In addition, in describing the present invention, when it is determined that the detailed description of the related well-known configuration or function may obscure the gist of the present invention, the detailed description thereof will be omitted.

Unless otherwise defined, all terms used in the present specification (including technical and scientific terms) may be used in a sense that can be commonly understood by those skilled in the art. In addition, the terms defined in the commonly used dictionaries are not ideally or excessively interpreted unless they are specifically defined clearly. The terminology used herein is for the purpose of describing particular embodiments only and is not intended to be limiting of the invention. In this specification, the singular also includes the plural unless specifically stated otherwise in the phrase.

In addition, in describing the component of this invention, terms, such as first, second, A, B, (a), (b), can be used. These terms are only for distinguishing the components from other components, and the nature or order of the components is not limited by the terms. If a component is described as being “connected,” “coupled” or “contacted” to another component, that component may be directly connected to or contacted with that other component, but it should be understood that another component also may be “connected,” “coupled” or “contacted” between each component.

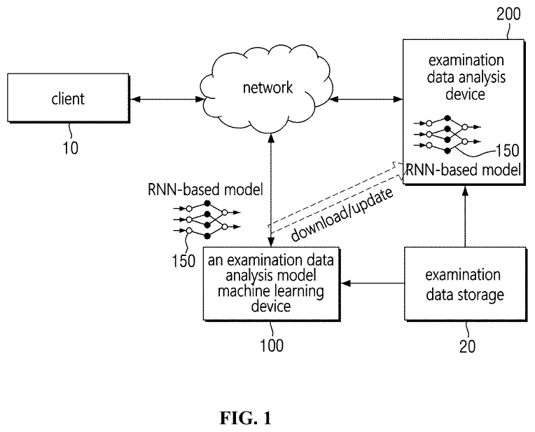

Hereinafter, some embodiments of the present invention will be described in detail with reference to the accompanying drawings. Referring to FIG. 1, a configuration and operation of a system for analyzing examination data according to an embodiment of the present inventive concept will be described.

The system for analyzing the examination data according to the present embodiment may include an examination data analysis model machine learning device 100. The system for analyzing the examination data according to the present embodiment may further include an examination data analysis device 200.

The examination data analysis model machine learning device 100 (hereinafter, abbreviated as “machine learning device”) receives and stores examination data from a device storing the examination data, such as an examination data storage 20. The machine learning device 100 configures an examination data analysis model by performing machine learning using the examination data as training data.

The examination data may satisfy a specific condition among examination data stored in the examination data storage 20, or may include tag information related to an output value of the examination data analysis model. For example, when the examination data analysis model is for predicting recurrence of a specific disease, the examination data may be composed of only a patient with a recurrence of the specific disease, or may include tag information on whether a disease recurs or not be added to the examination data. In other words, the machine learning device 100 may perform the machine learning in a supervised learning manner.

The examination data may not be data including one examination result, but may be time series data sequentially including examination results received by an examinee so far. For example, when the examination data analysis model is for predicting recurrence of a specific disease, the examination data may sequentially include results of examinations performed after a time point for curing the specific disease.

The examination data analysis model configured by the machine learning device 100 may be a model 150 based on a recurrent neural network (RNN) having high suitability for time series data. For example, the RNN-based model 150 may be a long short term memory (LSTM)-based model 150. The machine learning device 100 determines an optimized time interval of time slots included in a feature matrix. In addition, the machine learning device 100 performs hyper parameter optimization to determine an optimized look-back window size value which is applied in a machine learning process of the RNN-based model 150 proceeding using the feature matrix composed of the optimized time slots.

The look-back window size may be understood as a value indicating the number of time slots of past time series data considered in the learning process of the RNN-based model 150. For example, if the look-back window size is 10, a RNN-based neural network update using time series data of time slot n may be performed by referring to data from time slot n-10 to time slot n.

The determination of the optimized time interval and the look-back window size will be described later in detail.

The machine learning device 100 transmits model data (not shown) for configuring the RNN-based model 150 to the examination data analysis device 200. When the RNN-based model 150 is updated as the machine learning is performed again, the machine learning device 100 may transmit the updated model data to the examination data analysis device 200.

The examination data analysis device 200 receives latest examination data of the examinee from the examination data storage 20. The latest examination data may be composed of examination data of the last n times among examination results of the examinee or patient. The n may be a value corresponding to the optimized look-back window size. For example, the n may be the optimized look-back window size X 2 or double the optimized look-back window size.

The examination data analysis device 200 may configure the feature matrix using the latest examination data, and may input the feature matrix into the RNN-based model 150 to obtain prediction data related to a future prognosis of the examinee. The examination data analysis device 200 may generate report data by using the prediction data and then transmit the report data to a client 10. Transmission of the report data may be triggered by receiving a request from the client 10 by the examination data analysis device 200, or may be triggered by receiving a new examination data addition event of the examination data storage 20 by the examination data analysis device 200.

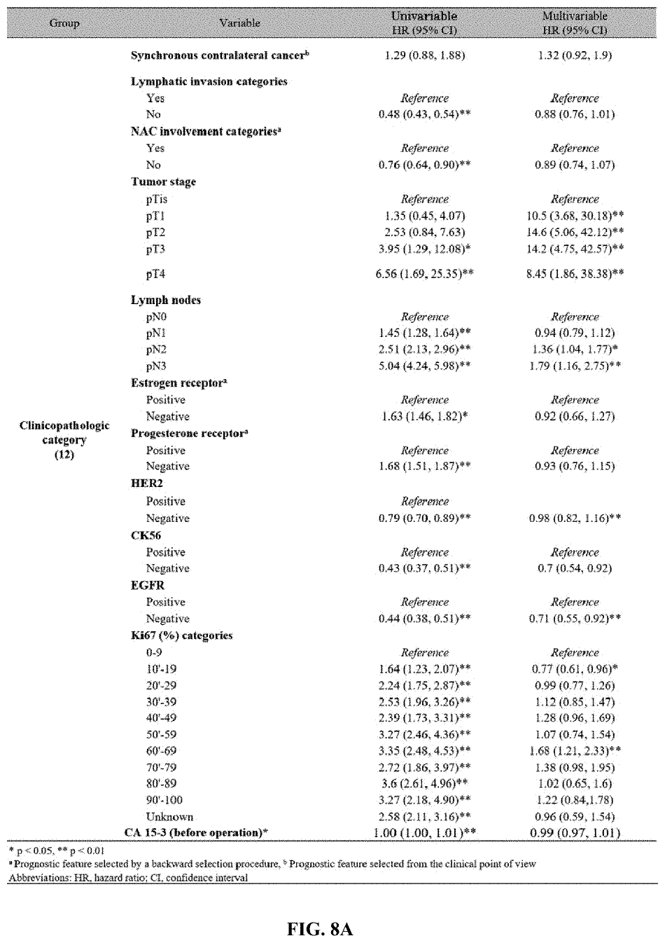

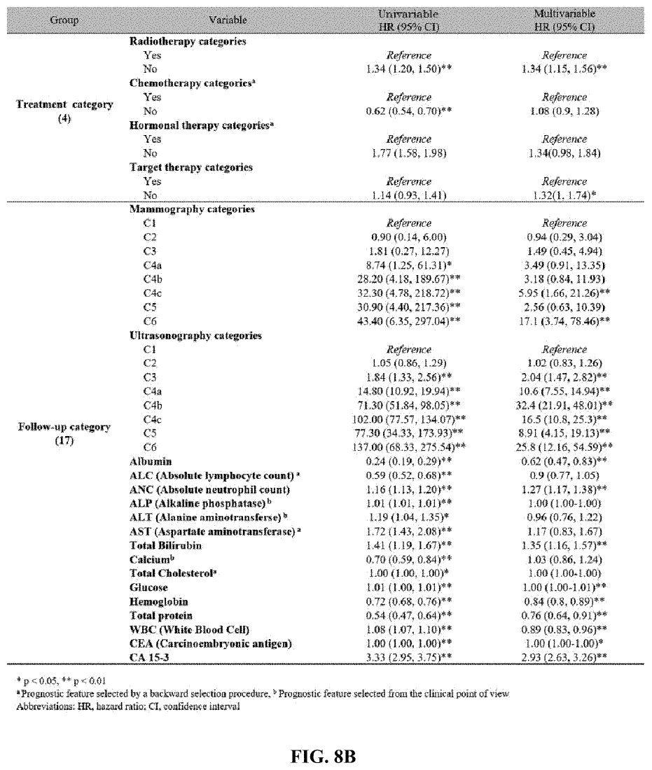

The RNN-based model 150 may be for predicting breast cancer recurrence. In other words, the RNN-based model 150 may receive a series of examination data after breast cancer surgery and output data related to a possibility of breast cancer recurrence. In order to configure the RNN-based model 150 for this purpose, results of optimal feature selection, which have been studied for a long period of time, are presented through FIGS. 8A to 8B. The results of the optimal feature selection will be described later in detail with reference to FIGS. 8A to 8B. However, the RNN-based model 150 presented in some embodiments of the present inventive concept is not limited to predicting recurrence of breast cancer, and further, is not limited to predicting recurrence of a specific disease. The RNN-based model 150 should be widely understood as analyzing examination data, which is time series data, that is, result data for each item of a series of medical examinations and medical tests performed on an examinee or patient.

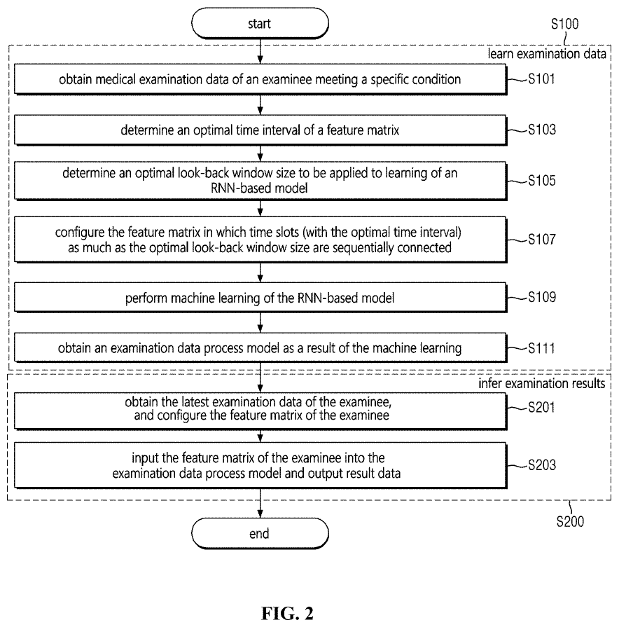

FIG. 2 illustrates a method for analyzing examination data according to another embodiment of the present inventive concept. The method according to the present embodiment is performed by a computing device. It is noted that all operations belonging to the method according to the present embodiment may be performed by one computing device, and some operations belonging to the method according to the present embodiment may be performed by a first computing device and other operations may be performed by a second computing device. For example, step (S100) of machine learning examination data may be performed by the examination data analysis model machine learning device 100 described with reference to FIG. 1, and the step (S200) of inferring examination result using an examination data analysis model may be performed by the examination data analysis device 100 described with reference to FIG. 1.

In step S101, medical examination data of an examinee meeting a specific condition is obtained. The specific condition will be determined according to a purpose of machine learning. For example, if the purpose of the machine learning is to predict recurrence of a specific disease, medical examination data of an examinee who has had the recurrence of the specific disease will be obtained.

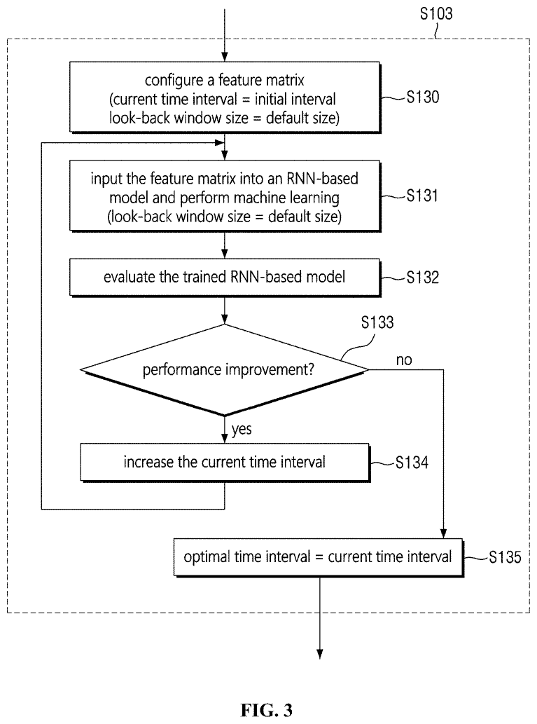

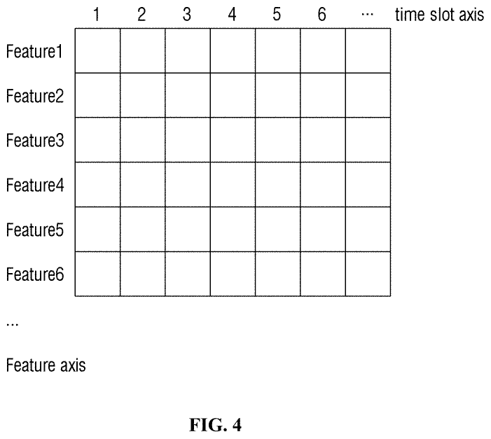

In step S103, an optimal time interval of a feature matrix is determined. As shown in FIG. 4, the feature matrix 30 for a specific examinee may be composed of a time slot axis (first axis) arranged in chronological order and a feature axis (second axis) in which different features are arranged. A time interval between each time slot must be the same. For example, if the time interval is 6 months, medical examination data at 6 months intervals will be included in the feature matrix 30. The optimal time interval determined in step S103 refers to a time interval in which performance of a model to be trained appears best among the time intervals between each time slot. Hereinafter, step S103 will be described in detail with reference to FIG. 3.

In step S130, a feature matrix is configured. Here, the time interval between each time slot is set to a predetermined initial interval, and a look-back window size is set to a predetermined default size. For example, the initial interval may be set to 1 month, and the default size may be set to 10. The default size of 10 means that when RNN-based machine learning is performed, data of the past 10 time slots are considered. For example, in this case, the time slot axis of the feature matrix may include 20 time slots (the number corresponding to double the look-back window size) with an interval of 1 month. In connection with step S103, the look-back window size is kept at the default size.

In step S131, the RNN-based machine learning is performed by inputting the feature matrix. Then, in step S132, the performance of the trained RNN-based model is evaluated. When the performance is evaluated, Harrell's C-index (concordance index) may be used as an index. A C-index value has a value between 0 and 1, and the closer to 1, the better performance is evaluated. In addition to the C-index value, an area under curve (AUC) value for model prediction may also be used for performance evaluation. Like the C-index value, the closer the AUC value is to 1, the better performance is evaluated.

In step S133, it is determined whether there is a performance improvement. If evaluation in step S132 is first evaluation, it will not be possible to evaluate whether the performance is improved. In this case, it is considered that there is an improvement in performance, and a current time interval is increased by one unit (e.g., 1 month) and steps S131 to S133 are repeated. If the evaluation of step S132 is not the first evaluation and there is an improvement in performance compared to a performance evaluation result of a previous step, the current time interval is increased by one unit and steps S131 to S133 are repeated. If the evaluation of step S132 is not the first evaluation and there is no improvement in performance compared to the performance evaluation result of the previous iteration, it is determined that the current time interval is an optimal time interval (S135).

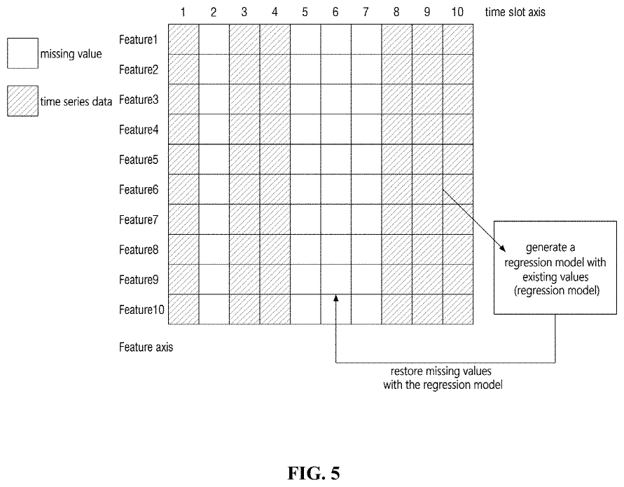

However, for example, if the current time interval is 2 months, there will not be many examinees who have undergone full medical examinations once every two months. Therefore, when the current time interval is short, many missing values will occur. In this case, as shown in FIG. 5, the missing value may be restored using a regression model generated using examination data of a time slot in which real time series data exists.

To abbreviate step S103 described with reference to FIG. 3, the following will be understood: due to a short time interval initially, many missing values are generated, and the performance of the trained RNN-based model is evaluated poorly; as the time interval increases, the performance of the RNN model is measured, and when the performance of the RNN model is no longer improved, the time interval at that iteration is the optimal time interval.

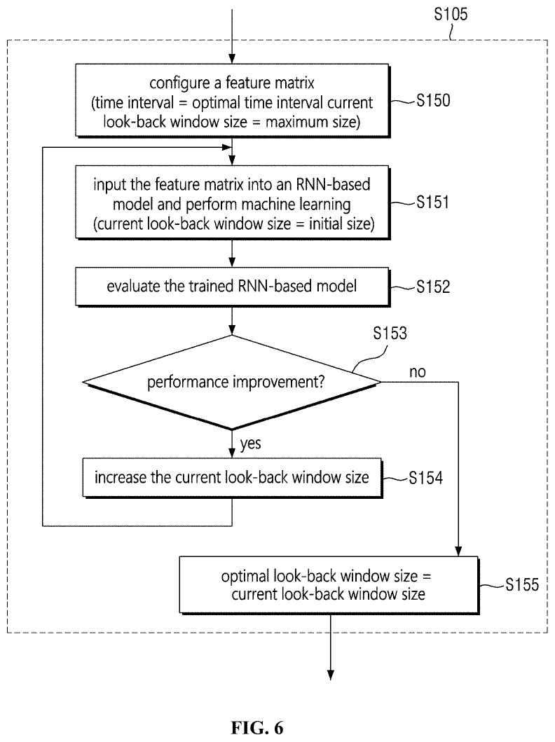

Referring now to step S105 in FIG. 2, when the feature matrix configured using the optimal time interval is input, an optimal look-back window size that enables an RNN-based model with optimal performance to be trained is now determined. A process of determining the optimal look-back window size is similar to that of step S103, and will now be described with reference to FIG. 6.

In step S150, a feature matrix is configured. Here, a time interval between each time slot is set to the optimal time interval, and a look-back window size is set to a predetermined maximum size. For example, the default size may be set to 100. The default size of 100 means that the feature matrix including a total of 100 time slots is configured in preparation for a situation in which a current look-back window size increases to 50. When configuring the feature matrix, the missing value restoration described with reference to FIG. 5 may be performed.

In step S151, RNN-based machine learning is performed by inputting the feature matrix. In the case of first machine learning in relation to step S105, the current look-back window size may be set to a predetermined initial size. The initial size may be, for example, two.

Then, in step S152, the performance of the trained RNN-based model is evaluated. When the performance is evaluated, Harrell's C-index (concordance index) may be used as an index. In addition to the C-index value, an area under curve (AUC) value for model prediction may also be used for performance evaluation.

In step S153, it is determined whether there is a performance improvement. If evaluation in step S152 is first evaluation, it will not be possible to evaluate whether the performance is improved. In this case, when it has considered that there is an improvement in performance, the current look-back window size is increased by one unit (e.g., 1) in step S154 and steps S151 to S153 are repeated. If the evaluation of step S152 is not the first evaluation and there is an improvement in performance compared to a performance evaluation result of a previous step, the current look-back window size is increased by one unit and steps S151 to S153 are repeated. If the evaluation of step S152 is not the first evaluation and there is no improvement in performance compared to the performance evaluation result of the previous step, it is determined that the current look-back window size is an optimal time interval (S155).

To abbreviate step S105 described with reference to FIG. 6, the following will be understood: due to a short look-back window size initially, the trained RNN-based model hardly reflects past time series data patterns, and the performance of the trained RNN-based model is evaluated poorly; as the look-back window size increases, the performance of the RNN model is measured, and when the performance of the RNN model is no longer improved, the current look-back window size is the optimal look-back window size. Even if the look-back window size is increased beyond the optimal look-back window size, computational resources and time are consumed without the added benefit in RNN model performance.

Referring now to step S107 in FIG. 2, the feature matrix is configured in which the time slots having the optimal time interval as well as the optimal look-back window are sequentially connected.

Referring now to step S109 in FIG. 2, the RNN-based model is machine-learned depending on the optimal look-back window size using the feature matrix having the optimal time interval. Here, the feature data may be divided into training data and test data at a ratio of, for example, 7:3. In addition, hyper parameter optimization may be additionally performed using the feature matrix composed of the training data. The hyper parameter optimization may be performed in a grid search manner. With respect to the RNN-based model trained using the feature matrix composed of the training data, the reliability may be evaluated by performing a validation using the feature matrix composed of the test data. The RNN-based model determined as having no problem through this validation will be used as a final examination data processing model (S111).

So far, the step (S100) of learning the examination data and generating a final examination data processing model has been described. Next, the step (S200) of inferring an examination result using the final examination data processing model will be described.

In step S201, the latest examination data of the examinee is obtained, and a feature matrix of the examinee is configured using the latest examination data. Here, the feature matrix is composed of time slots having the optimum time interval. A quantity of time slots included in the feature matrix is determined corresponding to the optimal look-back window size. For example, a quantity of time slots included in the feature matrix may be as much as twice the size of the optimal look-back window. For example, when the optimal time interval is 6 months and the optimal look-back window size is 5, the feature matrix may include 10 time slots at intervals of 6 months. This means that the last 10 examination data (6 months intervals) of the examinee are required for the configuration of the feature matrix.

In step S203, the feature matrix of the examinee is input to an examination data processing model, and examination result data (i.e. prognosis) is output based on an output value of the examination data processing model. The examination result data may be, for example, a report including a prediction result related to a specific disease recurrence of an examinee.

The output value of the examination data processing model may be an alpha value or a beta value of Weibull Distribution. The period in which recurrence is expected may be calculated using the alpha value and the beta value. In this regard, reference may be made to the published document <“WTTE-RNN: Weibull Time To Event Recurrent Neural Network”, Egil Martinsson, 2016>.

As described above, in some embodiments of the present inventive concept, the examination data processing model may be for obtaining a prediction or prognosis for breast cancer recurrence. The number of breast cancer patients is increasing every year. More than 20,000 people have been diagnosed with breast cancer every year since 2013. Breast cancer is one of the most important cancers, affecting more than 130,000 people worldwide. In the case of general solid cancer, it is judged to be cured after 5 years, whereas in the case of breast cancer, there are cases of continuous recurrence after 5 years, so a longer follow-up period is required than for other cancers.

Previously, a probability of recurrence was predicted based on a cancer stage and a subtype at the time of breast cancer surgery. Since the cancer stage and the subtype are not time-varying factors, it was difficult to accurately monitor the state that changes during the follow-up period. Therefore, for fear of recurrence and self-defense (i.e. defensive medicine), unnecessary examinations were continuously performed. For example, if the cancer stage and the subtype at the time of breast cancer surgery were in a bad state, no matter how good postoperative examination data comes out, it is necessary to continuously perform periodic examinations.

On the other hand, if breast cancer recurrence prediction is performed through the examination result analysis technology according to some embodiments of the present inventive concept, whenever an examination is taken, the probability of breast cancer recurrence is updated using an examinee's updated latest examination data. In addition, even if the examinee's examination period is not constant, pre-processing is performed to generate feature data, so the utilization is high.

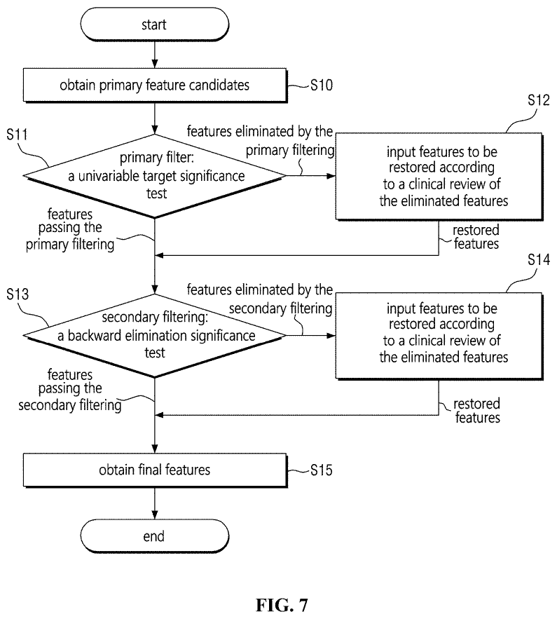

FIGS. 7 to 8B show a method for optimal feature selection that has been studied over a long period of time to configure an RNN-based model for predicting breast cancer recurrence, and its results.

As a result, 33 features related to breast cancer recurrence were selected. The process is shown in FIG. 7. 325 factors, including demographic, diagnosis, and other clinical characteristics, postoperative pathology results, treatment/surgical information, and time series follow-up results (blood examinations, mammography examinations) that may be obtained in breast cancer patients were selected as primary feature candidates (S10). For each of the primary feature candidates, primary filtering was performed through a univariable target significance test (S11). In other words, Hazard ratio values and variance significance tests for each factor were performed, and secondary feature candidates were selected. Some of the feature candidates that were eliminated from the primary filtering were added to the secondary feature candidates again according to a clinical review (S12).

Next, secondary filtering was performed on the secondary feature candidates using a backward elimination manner (S13). In the secondary filtering process, Akaike information criterion (AIC) model comparison was performed while subtracting the secondary feature candidates one by one, and some of the feature candidates that were eliminated from the secondary filtering were added as final features again according to the clinical review (S14).

FIGS. 8A to 8B illustrate 33 features used to predict breast cancer recurrence, which are 33 features derived through the process described with reference to FIG. 7.

The 33 features included 12 clinicopathologic features, 4 treatment-related features, and 17 follow-up features. In some embodiments, the feature used to predict whether breast cancer recurs may be some of the 33 features. In particular, the feature matrix may be configured using the 17 follow-up features shown in FIG. 8B.

A description will be given in connection with the interpretation of a univariable hazard ratio (HR) and multivariable hazard ratio (HR) shown in FIG. 8A. The univariable hazard ratio and multivariable hazard ratio mean that the higher the hazard ratio value, the higher the risk and the risk increases as the corresponding feature value increases. For example, for synchronous contralateral cancer features, the univariable hazard ratio is 1.29, which means that if a value of synchronous contralateral cancer increases by 1, an examinee's risk of recurrence of breast cancer increases by 1.29.

However, the hazard ratio value cannot be the same for all examinees. Accordingly, the distribution of hazard ratio values according to a 95% confidence interval is also shown in FIGS. 8A to 8B. For example, for synchronous contralateral cancer features, the univariable hazard ratio is 1.29, and the distribution of hazard ratio values according to the 95% confidence interval is 0.88 to 1.88.

The univariable hazard ratio is a hazard ratio derived by analyzing the impact of one variable, and the multivariable hazard ratio is a result of analyzing the impact of each variable through combination.

In addition, in the case of features classified by status, such as steps not values, positive/negative, category, etc., the hazard ratio value refers to a degree of risk compared to the case in which a state of the corresponding feature is indicated by ‘reference’ when the status of the feature is not in the status indicated as ‘Reference.’ For example, for the univariable hazard ratio of the feature ‘lymphatic invasion categories,’ if the feature ‘lymphatic invasion categories’ is ‘No,’ the hazard ratio is interpreted to be 0.48 compared to ‘Yes.’ In other words, when the status of the feature ‘lymphatic invasion categories’ is ‘No,’ it may be interpreted that the hazard ratio of recurrence of breast cancer is low.

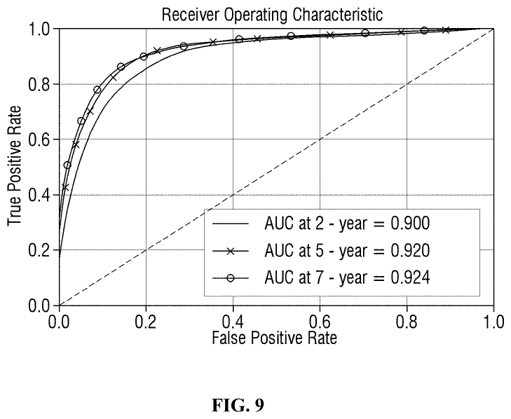

FIG. 9 shows the accuracy of predicting breast cancer recurrence of the RNN model trained according to step S100 of FIG. 2 using the feature matrix configured using the 33 features related to breast cancer recurrence. As both a CI score and an AUC score are very close to 1, it may be seen that the RNN model trained according to some embodiments of the present inventive concept may perform breast cancer recurrence prediction with high accuracy.

The technical teaching of the present disclosure described with reference to FIGS. 1 to 9 may be implemented as computer readable codes on a computer readable medium. The computer-readable recording medium may be, for example, a removable recording medium (CD, DVD, Blu-ray disc, USB storage device, removable hard disk) or a fixed recording medium (ROM, RAM, computer equipped hard disk). The computer program recorded on the computer-readable recording medium may be transmitted to other computing device a network such as the Internet and installed in the other computing device, thereby being used in the other computing device.

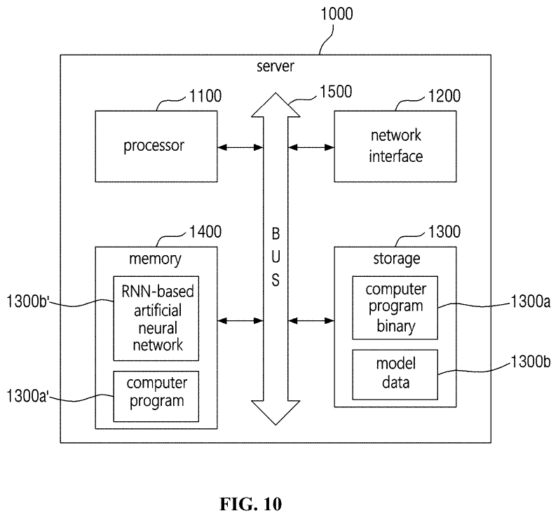

Hereinafter, an exemplary computing device capable of implementing a device according to some embodiments of the present inventive concept will be described with reference to FIG. 10. FIG. 10 is an exemplary hardware configuration diagram illustrating a server 1000.

As shown in FIG. 10, the server 1000 may include a one or more processors 1100, a bus 1500, a network interface 1200, a memory 1400 to load a computer program 1300a executed by the processor 1100, and a storage 1300 to store the computer program 1300a. However, FIG. 10 illustrates only components related to an embodiment of the present disclosure. Accordingly, it will be appreciated by those skilled in the art that the present disclosure may further include other general purpose components in addition to the components illustrated in FIG. 10.

The processor 1100 controls overall operations of each component of the computing device 1000. The processor 1100 may be configured to include at least one of a Central Processing Unit (CPU), a Micro Processor Unit (MPU), a Micro Controller Unit (MCU), a Graphics Processing Unit (GPU), or any type of processor well known in the art. Further, the processor 1100 may perform calculations on at least one application or program for executing a method/operation according to various embodiments of the present disclosure. The computing device 1000 may have one or more processors.

The memory 1400 stores various types of data, instructions, and/or information. The memory 1400 may load one or more computer program binaries 1300a from the storage 1300 in order to execute methods/operations according to various embodiments of the present disclosure. An example of the memory 1400 may be a RAM, but is not limited thereto.

The bus 1500 provides communication between components of the computing device 1000. The bus 1500 may be implemented as various types of bus such as an address bus, a data bus and a control bus.

The network interface 1200 supports wired/wireless Internet communication of the server 1000. The network interface 1200 may support various communication methods other than Internet communication. To this end, the network interface 1200 may include a communication module well known in the technical field of the present inventive concept.

The storage 1300 may non-temporarily store one or more computer programs 1300a. In addition, the storage 1300 may further store model data 1300b.

The computer program 1300a may include one or more instructions in which methods/actions according to various embodiments of the present inventive concept are implemented. When the computer program 1300a is loaded into the memory 1400, the processor 1100 executes the one or more instructions to perform methods/operations according to various embodiments of the present disclosure. When the server 1000 is a device that performs the role of the examination data analysis model machine learning device 100 described with reference to FIG. 1, the computer program 1300a may include an instruction for setting a time interval applied to a time axis of a two-dimensional feature matrix comprising the time axis and each feature as a predetermined initial interval to configure the feature matrix, and obtaining a first performance evaluation result of a recurrent neural network (RNN)-based model trained by using the feature matrix, an instruction for first repeating increasing the time interval and then obtaining the first performance evaluation result of the RNN-based model trained by using the feature matrix according to the increased time interval until the first performance evaluation result is no longer improved, an instruction for determining the time interval that is last increased in the instruction for first repeating as an optimal time interval, an instruction for configuring the feature matrix with the time axis according to the optimal time interval, and by using the feature matrix, setting a look-back window size to a predetermined initial size to obtain a performance evaluation result of the trained RNN-based model, an instruction for second repeating increasing the look-back window size and then obtaining a second performance evaluation result of the RNN-based model trained according to the increased look-back window size until the second performance evaluation result is no longer improved, an instruction for determining the look-back window size that is last increased in the instruction for second repeating as an optimal look-back window size, and an instruction for training the RNN-based model according to the optimal look-back window size by using the feature matrix having the optimal time interval. Data representing the finally learned RNN-based artificial neural network may be packaged and stored as model data 1300b in the storage 1300. The model data 1300b may be transmitted to an external device through the network interface 1200.

When the RNN-based model packaged as model data 1300b and stored in the storage 1300 outputs data related to breast cancer recurrence prediction, the features included in the feature matrix may include at least some of mammography category, ultrasonography category, albumin level, absolute lymphocyte count (ALC) level, absolute neutrophil count (ANC) level, alkaline phosphatase (ALP) level, alanine aminotransferase (ALT) level, aspartate aminotransferase (AST) level, total bilirubin level, calcium level, total cholesterol level, glucose level, hemoglobin level, total protein level, white blood cell (WBC) level, carcinoembryonic antigen (CEA) level, CA15-3 level, radiotherapy category, chemotherapy category, hormonal therapy category, target therapy category after breast cancer surgery, synchronous contralateral cancer category, whether there is lymphatic invasion or not, whether there is NAC involvement or not, tumor stage, lymph nodes, whether it is estrogen receptor positive or not, whether it is progesterone receptor positive or not, whether it is HER2 positive or not, whether it is CK56 positive or not, whether it is EGFR positive or not, Ki67(%) category and preoperative CA 15-3 level.

When the server 1000 is a device that performs the role of the examination data analysis device 200 described with reference to FIG. 1, the computer program 1300a may include an instruction for obtaining latest examination data of an examinee, and configuring a feature matrix by using the latest examination data, and an instruction for inputting the feature matrix into an RNN-based model, and generating data for predicting breast cancer recurrence of the examinee by using an output value of the RNN-based model, The RNN-based model may be configured on a memory 1300b′ based on model data 1300b stored in the storage 1300.

When the server 1000 is a device that performs the role of the examination data analysis device 200 described with reference to FIG. 1 and is a device that predicts breast cancer recurrence, the features included in the feature matrix may include at least some of mammography category, ultrasonography category, albumin level, absolute lymphocyte count (ALC) level, absolute neutrophil count (ANC) level, alkaline phosphatase (ALP) level, alanine aminotransferase (ALT) level, aspartate aminotransferase (AST) level, total bilirubin level, calcium level, total cholesterol level, glucose level, hemoglobin level, total protein level, white blood cell (WBC) level, carcinoembryonic antigen (CEA) level, CA 15-3 level, radiotherapy category, chemotherapy category, hormonal therapy category, target therapy category after breast cancer surgery, synchronous contralateral cancer category, whether there is lymphatic invasion or not, whether there is NAC involvement or not, tumor stage, lymph nodes, whether it is estrogen receptor positive or not, whether it is progesterone receptor positive or not, whether it is HER2 positive or not, whether it is CK56 positive or not, whether it is EGFR positive or not, Ki67(%) category and preoperative CA 15-3 level.

The methods according to the embodiments described above can be performed by the execution of a computer program implemented as computer-readable code. The computer program may be transmitted from a first computing device to a second computing device through a network such as the Internet and may be installed in the second computing device and used in the second computing device. Examples of the first computing device and the second computing device include fixed computing devices such as a server, a physical server belonging to a server pool for a cloud service, and a desktop PC.

The computer program may be stored in a non-transitory recording medium such as a DVD-ROM or a flash memory.

The concepts of the inventive concept described above can be embodied as computer-readable code on a computer-readable medium. The computer-readable medium may be, for example, a removable recording medium (a CD, a DVD, a Blu-ray disc, a USB storage device, or a removable hard disc) or a fixed recording medium (a. ROM, a RAM, or a computer-embedded hard disc). The computer program recorded on the computer-readable recording medium may be transmitted to another computing apparatus via a network such as the Internet and installed in the computing apparatus. Hence, the computer program can be used in the computing apparatus.

Although operations are shown in a specific order in the drawings, it should not be understood that desired results can be obtained when the operations must be performed in the specific order or sequential order or when all of the operations must be performed, in certain situations, multitasking and parallel processing may be advantageous. According to the above-described embodiments, it should not be understood that the separation of various configurations is necessarily required, and it should be understood that the described program components and systems may generally be integrated together into a single software product or be packaged into multiple software products.

While the present inventive concept has been particularly illustrated and described with reference to exemplary embodiments thereof, it will be understood by those of ordinary skill in the art that various changes in form and detail may be made therein without departing from the spirit and scope of the present inventive concept as defined by the following claims. The exemplary embodiments should be considered in a descriptive sense only and not for purposes of limitation.

What is claimed is:

1. A method for processing medical examination data, wherein the medical examination data is time series data, and the method is performed by a computing device, the method comprising:
providing a two-dimensional feature matrix having a time axis and a feature axis, the feature axis representing a plurality of features;
setting a time interval applied to the time axis of the feature matrix to a predetermined initial interval to configure the feature matrix;
obtaining a first performance evaluation result of a recurrent neural network (RNN)-based model trained by using the feature matrix;
first repeating increasing the time interval and then obtaining the first performance evaluation result of the RNN-based model trained by using the feature matrix according to the increased time interval until the first performance evaluation result is no longer improved;
determining the time interval that is last increased in the first repeating as an optimal time interval;
configuring the feature matrix with the time axis according to the optimal time interval, and by using the feature matrix, setting a look-back window size to a predetermined initial size to obtain a performance evaluation result of the trained RNN-based model;
second repeating increasing the look-back window size and then obtaining a second performance evaluation result of the RNN-based model trained according to the increased look-back window size until the second performance evaluation result is no longer improved;
determining the look-back window size that is last increased in the second repeating as an optimal look-back window size; and
training the RNN-based model according to the optimal look-back window size by using the feature matrix having the optimal time interval.

2. The method of claim 1, wherein the obtaining the first performance evaluation result comprises training the RNN-based model by setting the look-back window size to a predetermined default size; and
the first repeating comprises training the RNN-based model by setting the look-back window size to the predetermined default size.

3. The method of claim 1, wherein the obtaining the first performance evaluation result comprises filling a missing value according to the initial interval by using a regression model generated with data of a time slot in which the medical examination data exists; and
the first repeating comprises filling the missing value according to the initial interval by using the regression model generated with data of the time slot in which the medical examination data exists.

4. The method of claim 1, wherein the RNN-based model outputs data related to prediction of breast cancer recurrence after breast cancer surgery; and
the features included within the feature matrix comprises mammography category, ultrasonography category, albumin level, absolute lymphocyte count (ALC) level, absolute neutrophil count (ANC) level, alkaline phosphatase (ALP) level, alanine aminotransferase (ALT) level, aspartate aminotransferase (AST) level, total bilirubin level, calcium level, total cholesterol level, glucose level, hemoglobin level, total protein level, white blood cell (WBC) level, carcinoembryonic antigen (CEA) level, and CA 15-3 level.

5. The method of claim 4, wherein the features included within the feature matrix further comprises radiotherapy category, chemotherapy category, hormonal therapy category, and target therapy category after breast cancer surgery.

6. The method of claim 4, wherein the features included within the feature matrix further comprises synchronous contralateral cancer category, whether there is lymphatic invasion or not, whether there is NAC involvement or not, tumor stage, lymph nodes, whether it is estrogen receptor positive or not, whether it is progesterone receptor positive or not, whether it is HER2 positive or not, whether it is CK56 positive or not, whether it is EGFR positive or not, Ki67(%) category, and preoperative CA 15-3 level.

7. The method of claim 1, wherein the RNN-based model outputs data related to prediction of breast cancer recurrence after breast cancer surgery;
the method further comprises, after training the RNN-based model according to the optimal look-back window size by using the feature matrix having the optimal time interval, inputting latest examination data of an examinee into the trained RNN-based model and obtaining data for predicting breast cancer recurrence, and
the latest examination data comprise the number of latest examination data corresponding to the optimal look-back window size of the examinee.

8. A method for processing medical examination data, wherein medical examination data is time series data, and the method is performed by a computing device, the method comprising:
obtaining latest examination data of an examinee;
configuring a feature matrix by using the latest examination data, the feature matrix including a plurality of features;
inputting the feature matrix into an RNN-based model; and
generating data for predicting breast cancer recurrence of the examinee by using an output value of the RNN-based model,
wherein the features included within the feature matrix comprises mammography category, ultrasonography category, albumin level, absolute lymphocyte count (ALC) level, absolute neutrophil count (ANC) level, alkaline phosphatase (ALP) level, alanine aminotransferase (ALT) level, aspartate aminotransferase (AST) level, total bilirubin level, calcium level, total cholesterol level, glucose level, hemoglobin level, total protein level, white blood cell (WBC) level, carcinoembryonic antigen (CEA) level, CA 15-3 level, radiotherapy category, chemotherapy category, hormonal therapy category, target therapy category after breast cancer surgery, synchronous contralateral cancer category, whether there is lymphatic invasion or not, whether there is NAC involvement or not, tumor stage, lymph nodes, whether it is estrogen receptor positive or not, whether it is progesterone receptor positive or not, whether it is HER2 positive or not, whether it is CK56 positive or not, whether it is EGFR positive or not, Ki67(%) category, and preoperative CA 15-3 level.

9. The method of claim 8, wherein a time axis of the feature matrix is divided into a plurality of time slots, each having a predetermined optimal time interval, the time slots being sequentially connected by a number corresponding to a predetermined optimal look-back window size; and
configuring the feature matrix comprises filling a missing value due to not performing a medical examination corresponding to one of the time slots of the feature matrix by using a regression model generated using data of the one of the time slots in which the medical examination data exists.

10. An apparatus to process examination data, comprising:
a processor;
a memory; and
a computer program loaded into the memory and executed by the processor, the computer program comprising:
an instruction to configure a feature matrix having a time axis and a feature axis having a plurality of features by setting a time interval applied to a time axis to a predetermined initial interval;
an instruction to obtain a first performance evaluation result of a recurrent neural network (RNN)-based model trained by using the feature matrix;
an instruction for first repeating increasing the time interval and then obtaining the first performance evaluation result of the RNN-based model trained by using the feature matrix according to the increased time interval until the first performance evaluation result is no longer improved;
an instruction for determining the time interval that is last increased in the instruction for first repeating as an optimal time interval;
an instruction for configuring the feature matrix with the time axis according to the optimal time interval;
an instruction for setting a look-back window size to a predetermined initial size to obtain a performance evaluation result of the trained RNN-based model;
an instruction for second repeating increasing the look-back window size and then obtaining a second performance evaluation result of the RNN-based model trained according to the increased look-back window size until the second performance evaluation result is no longer improved;
an instruction for determining the look-back window size that is last increased in the instruction for second repeating as an optimal look-back window size; and
an instruction for training the RNN-based model according to the optimal look-back window size by using the feature matrix having the optimal time interval.

11. The apparatus of claim 10, wherein the instruction for obtaining the first performance evaluation result comprises an instruction for training the RNN-based model by setting the look-back window size to a predetermined default size; and
the instruction for first repeating comprises an instruction for training the RNN-based model by setting the look-back window size to the predetermined default size.

12. The apparatus of claim 10, wherein the instruction for obtaining the first performance evaluation result comprises an instruction for filling a missing value according to the initial interval by using a regression model generated with data of a time slot in which the medical examination data exists; and
the instruction for first repeating comprises the instruction for filling the missing value according to the initial interval by using the regression model generated with data of the time slot in which the medical examination data exists.

13. The apparatus of claim 10, wherein the RNN-based model outputs data related to prediction of breast cancer recurrence after breast cancer surgery; and
the features included within the feature matrix comprise mammography category, ultrasonography category, albumin level, absolute lymphocyte count (ALC) level, absolute neutrophil count (ANC) level, alkaline phosphatase (ALP) level, alanine aminotransferase (ALT) level, aspartate aminotransferase (AST) level, total bilirubin level, calcium level, total cholesterol level, glucose level, hemoglobin level, total protein level, white blood cell (WBC) level, carcinoembryonic antigen (CEA) level, and CA 15-3 level.

14. The apparatus of claim 13, wherein the features included within the feature matrix further comprise radiotherapy category, chemotherapy category, hormonal therapy category, and target therapy category after breast cancer surgery.

15. The apparatus of claim 13, wherein the features included within the feature matrix further comprise synchronous contralateral cancer category, whether there is lymphatic invasion or not, whether there is NAC involvement or not, tumor stage, lymph nodes, whether it is estrogen receptor positive or not, whether it is progesterone receptor positive or not, whether it is HER2 positive or not, whether it is CK56 positive or not, whether it is EGFR positive or not, Ki67(%) category, and preoperative CA 15-3 level.

16. The apparatus of claim 10, wherein the RNN-based model outputs data related to prediction of breast cancer recurrence after breast cancer surgery; and
the computer program further comprises, after the instruction for training the RNN-based model according to the optimal look-back window size by using the feature matrix having the optimal time interval, an instruction for inputting latest examination data of an examinee into the trained RNN-based model and an instruction for obtaining data for predicting breast cancer recurrence; and
the latest examination data comprises the number of latest examination data corresponding to the optimal look-back window size of the examinee.
